(19) United States
(12) Patent Application Publication
(10) Pub. No.: US 2008/0172036 A1
(43) Pub. Date: Jul. 17, 2008

(54) Guide catheter and method for advancing a guide catheter in a vessel

(76) Inventor: Adam Stys, Sioux Falls, SD (US)

Correspondence Address: Roeder & Broder LLP, 5560 Chelsea Avenue, La Jolla, CA, 92037, US

(21) Appl. No.: 12/006,989

(22) Filed: Jan. 8, 2008

Related U.S. Application Data
(60) Provisional application No. 60/880,121, filed on Jan. 12, 2007.

(51) Int. Cl. A61M 25/092; A61M 25/088

(52) U.S. Cl. 604/525; 604/523

(57) ABSTRACT

A guide catheter (16) for movement within a vessel (12) includes a tubular catheter assembly (20) having an exposed end (16B) that is positioned outside the vessel (12), and a catheter tip (16A) that is movable within the vessel (12) with manipulation of the exposed end (16B). In certain embodiments, at least one of a stiffness and a shape of the catheter tip (16A) can be adjusted between a first configuration (18) and a second configuration (318) with the exposed end (16B). As a result of this design, the physician can move the distal catheter tip (16A) with less resistance through narrow, tortuous vessels (12), the physician can accurately position the distal catheter tip (16A) in the ostium (12C), and the distal catheter tip (16A) is more likely to remain in position in the ostium (12C) when a guide wire and other treatment devices are directed through the guide catheter (16).

### RELATED APPLICATION

The application claims priority on U.S. Provisional Application No. 60/880,121, entitled “GUIDE CATHETER AND METHOD FOR ADVANCING A GUIDE CATHETER IN A VESSEL” filed on Jan. 12, 2007. The contents of U.S. Provisional Application No. 60/880,121 are incorporated herein by reference.

### BACKGROUND

The process of atherosclerosis causes fatty deposits (plaque) to accumulate in the walls of arteries of a heart. As the process becomes more advanced, the fatty deposits begin to encroach on the lumen of the artery, resulting in blockages (stenosis) of varying degrees and reduction in blood flow. One treatment of such blockages is a procedure commonly referred to as angioplasty. A typical angioplasty procedure includes (i) inserting a sheath into a blood vessel in the groin or arm, (ii) inserting a guide catheter into a lumen of the sheath, (iii) moving a catheter tip of the guide catheter through the blood vessel into the aorta of the heart until the catheter tip is positioned in an ostium of one of the coronary arteries, (iv) moving a guide wire through the guide catheter until the guide wire is positioned in the coronary artery past the blockage, (v) moving a balloon catheter through the guide catheter and over the guide wire until the balloon is positioned at the blockage, (vi) expanding the balloon to open the blockage, (vii) deflating the balloon, and (viii) sequentially removing the balloon catheter, the guide wire, the guide catheter, and the sheath from the patient.

Additionally, prior to removing the guide wire, the guide catheter, and the sheath, a stent can be moved through the guide catheter and over the guide wire until the stent is positioned at the site of the blockage. Subsequently, the stent can be expanded against the inner wall of the artery to support the inner wall. In certain patients, the stent reduces the rate of renarrowing at the treatment site.

Typically, the physician gently advances and rotates an exposed end of the guide catheter that is positioned outside the patient to move the catheter tip through the vessel and position the catheter tip in the ostium of the coronary artery. Unfortunately, individual variations in arota size, coronary arteries sizes and take offs, calcification and tortuosity in the vessel in certain patients makes placement of catheter tip into the coronary ostium very difficult.

Further, movement of the guide wire, the balloon catheter and/or the stent through the guide catheter into the coronary artery can cause the guide catheter to become unseated from and back out of the ostium of the coronary artery. If catheter tip becomes unseated, the catheter tip must be repositioned in the ostium. This can greatly complicate the procedure being performed on the patient.

### SUMMARY

The present invention is directed to a guide catheter for movement within a vessel. The guide catheter includes a tubular catheter assembly having an exposed end that is positioned outside the vessel, and a catheter tip that is movable within the vessel with manipulation of the exposed end. In certain embodiments, at least one of a stiffness and a shape of the catheter tip (sometimes referred to as the “distal end’) can be adjusted between a first configuration and a second configuration with the exposed end. As a result of this design, the physician can position the distal catheter tip more accurately and easily in the ostium. Further, the distal catheter tip is more likely to remain in position in the ostium when a guide wire and other treatment devices are directed through the guide catheter, as the guide catheter stiffness can be adjusted. Typically, the stiffer the guide catheter, the easier it is to position the devices in a coronary artery through it. This reduces trauma on the patient and increases the likelihood that the procedure performed on the patient will be successful.

In one non-exclusive example, the shape of the catheter tip is adjustable between a tip radius of approximately 0.5 centimeters and 7 centimeters; and/or a stiffness of the catheter tip is adjustable between 1 and 100 percent. In one embodiment, both the stiffness and the shape of the catheter tip can be adjusted with the exposed end.

In one embodiment, the guide catheter assembly includes a first catheter and a second catheter that can be moved together within the vessel. The first catheter includes a tubular first distal end that is sized and shaped to be positioned within the vessel. The second catheter includes a tubular second distal end that is sized and shaped to be positioned within in the first catheter. In one embodiment, relative movement of the catheters changes at least one of the stiffness and the shape of the catheter tip. For example, relative movement of the catheters along an axis can change at least one of the stiffness and the shape of the catheter tip.

In one embodiment, the first distal end is curved at a first distal curve and the second distal end is curved at a second distal curve. For example, the first distal curve can be less than the second distal curve. Stated in another fashion, the first distal curve can have a first radius that is greater than a second radius of the second distal curve. In one non-exclusive example, the first radius is between approximately 6 centimeters and 12 centimeters, and the second radius is between approximately 3 centimeters and 6 centimeters. With this design, relative movement of the catheters changes both the stiffness and the shape of the catheter tip.

Additionally, the guide catheter can include a rotation inhibitor that inhibits relative rotation between the first distal end and the second distal end. With this design, relative movement between the first catheter and the second catheter is limited to sliding along a first axis. This simplifies the control of the stiffness and the shape of the catheter tip.

The present invention is also directed to method for moving a guide catheter in a vessel of a mammal. The method can include the steps of (i) providing a tubular catheter assembly having an exposed end that is positioned outside the vessel, and a catheter tip that is movable within the vessel with manipulation of the exposed end; and (ii) selectively adjusting at least one of a stiffness and a shape of the catheter tip while the catheter tip is positioned in the vessel.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a simplified illustration of a portion of a heart, a portion of a sheath, and a portion of a guide catheter positioned in the heart;

FIG. 1B is a simplified, cut-away, side illustration of a portion of a vessel of the heart, and a portion of the guide catheter in a first configuration positioned in the vessel;

FIG. 2A is a simplified side view of the guide catheter of FIG. 1 in the first configuration;

FIG. 2B is a simplified exploded view of the guide catheter of FIG. 1;

FIG. 2C is a simplified cut-away view of the guide catheter of FIG. 1 in the first configuration;

FIG. 3 is a simplified cut-away view of the guide catheter in a second configuration;

FIG. 4 is a simplified cut-away view of the guide catheter in a third configuration;

FIG. 5 is a simplified end view of a guide catheter having features of the present invention;

FIG. 6A is a simplified exploded view of yet another embodiment of the guide catheter;

FIG. 6B is a simplified side view of the guide catheter of FIG. 6A in a first configuration; and

FIG. 6C is a simplified side view of the guide catheter of FIG. 6A is a second configuration.

### DESCRIPTION

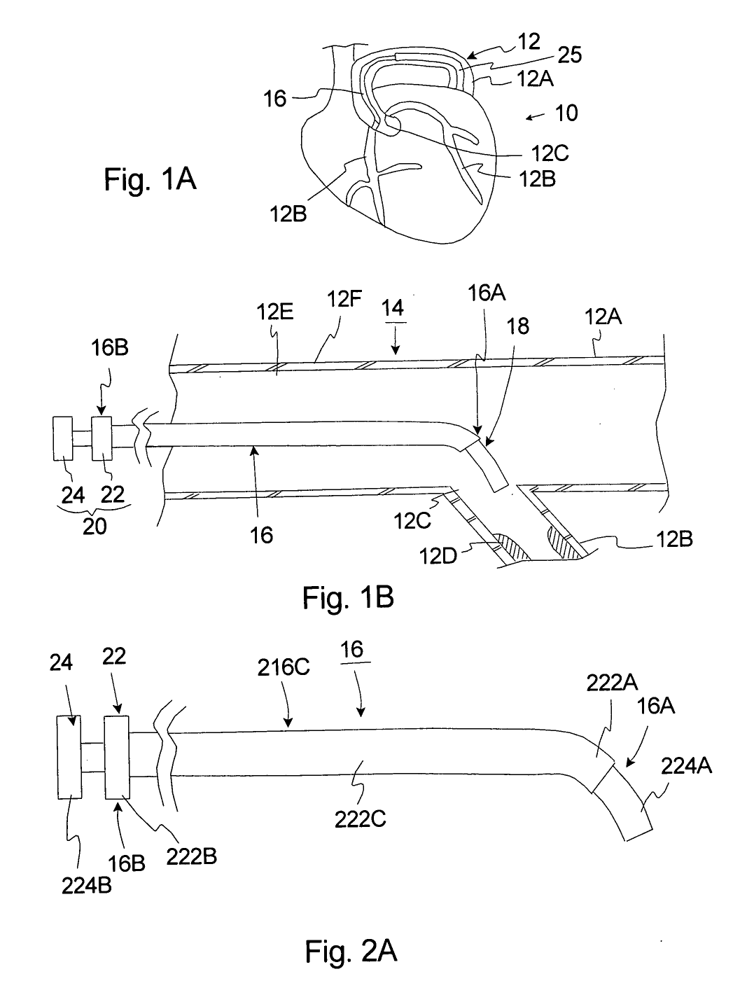

FIG. 1A is a simplified illustration of a portion of a heart 10 and FIG. 1B is a simplified, cut-away side illustration of a portion of a vessel 12 from the heart 10 of a patient 14 and a portion of a guide catheter 16 in a first configuration 18 positioned in the ostium of the vessel 12. In FIGS. 1A and 1B, the vessel 12 of the heart 10 includes a main branch 12A, and one or more side branches 12B, each having an ostium 12C and a treatment site 12D (partly illustrated in FIG. 1B). As an overview, in certain embodiments, the guide catheter 16 is uniquely designed so that a shape and/or a stiffness of a distal catheter tip 16A of the guide catheter 16 can be selectively adjusted while the guide catheter 16 is positioned in the vessel 12. As a result of this design, the physician (not shown) can move the distal catheter tip 16A with less resistance through narrow, tortuous vessels 12, the physician can accurately position (“park”) the distal catheter tip 16A in the ostium 12C of the vessel 12, and the distal catheter tip 16A is more likely to remain in position in this ostium 12C when a guide wire (not shown) and other treatment devices (not shown) are directed through the guide catheter 16. Further, with this design, the guide wire and/or other treatment devices move easier within the guide catheter 16. This reduces trauma on the patient and increases the likelihood that the procedure performed on the patient 14 will be successful. This also allows the physician to use a greater variety of guide wires and/or other treatment devices.

The type of vessel 12 and treatment site 12D can vary. For example, the vessel 12 can be an artery of a mammal, such as a human being. Alternatively, for example, the vessel 12 can be another body passageway in the vascular system or an organ. In one embodiment, the main branch 12A and the side branch 12B each include a vessel lumen 12E and a vessel wall 12F. The location of the side branch 12B relative to the main branch 12A can vary. In FIGS. 1A and 1B, the main branch 12A is an aorta of the heart 10, and the side branch 12B is the right coronary artery of the heart 10 of the patient 14. Alternatively, for example, the side branch 12B that is being treated can be the left main coronary artery.

The location and type of the treatment site 14 can vary according to the needs of the patient. For example, in FIG. 1, the treatment site 14 is in the side branch 12B. Further, in this embodiment, the treatment site 12D includes a fatty deposit of material (not shown), e.g. plaque, on the inner lining of the vessel wall 12F. In this embodiment, an angioplasty procedure may be performed on the treatment site 12D and/or a stent (not shown) can be positioned at the treatment site 12D. Alternatively, another type of treatment can be performed on the treatment site 12D.

The guide catheter 16 can be introduced into the patient 14 wherever it is most convenient to do so. For example, the guide catheter 16 can be inserted into a blood vessel in the groin (not shown) or arm (not shown) of the patient 14.

As illustrated in FIG. 1B, the guide catheter 16 additionally includes an exposed end 16B that is opposite from the catheter tip 16A and that is positioned outside the patient 14. The physician moves the exposed end 16B to navigate the catheter tip 16A through the vessel 12. Additionally, as provided herein, the physician moves the exposed catheter end 16B to selectively adjust the shape and/or a stiffness of the distal catheter tip 16A while the guide catheter 16 is positioned in the vessel 12.

FIG. 1B illustrates the guide catheter 16 has been moved in the vessel 12 so that the distal catheter tip 16A is positioned in the ostium 12C in the side branch 12B. It should be noted that the shape of the distal catheter tip 16A can be adjusted to facilitate pushing the distal catheter tip 16A through the vessel 12 and facilitate accurate positioning of the distal catheter tip 16A in the ostium 12C. Further, the shape and stiffness of the distal catheter tip 16A can be adjusted so that the distal catheter tip 16A will remain positioned in the ostium 12C during movement of a guide wire and/or other treatment device through the guide catheter 16.

In one embodiment, the guide catheter 16 is a catheter assembly 20 having a tubular first catheter 22 and a tubular second catheter 24 that are designed to be moved together within the vessel 12. Further, the catheters 22, 24 are designed to move relative to each other to selectively adjust and alter the stiffness and/or the shape of the catheter tip 16A.

FIG. 1A illustrates that a sheath 25 has been positioned in a portion of the main branch 12A. The guide catheter 16 is inserted into the sheath 25 and the sheath 25 is used to guide the guide catheter 16 to the heart 10.

With the present invention, the catheter tip 16A is parked in the ostium 12C and intervention is done distal to the catheter tip 16A. While the guide catheter 16 is parked in the main branch 12A with the catheter tip 16A at the ostium 12C, the shape and stiffness of the guide catheter 16 defines its stability and whether the catheter tip 16A will remain at the ostium 12C in spite of backing away forces from pushing in the equipment deep into the side branch 12B.

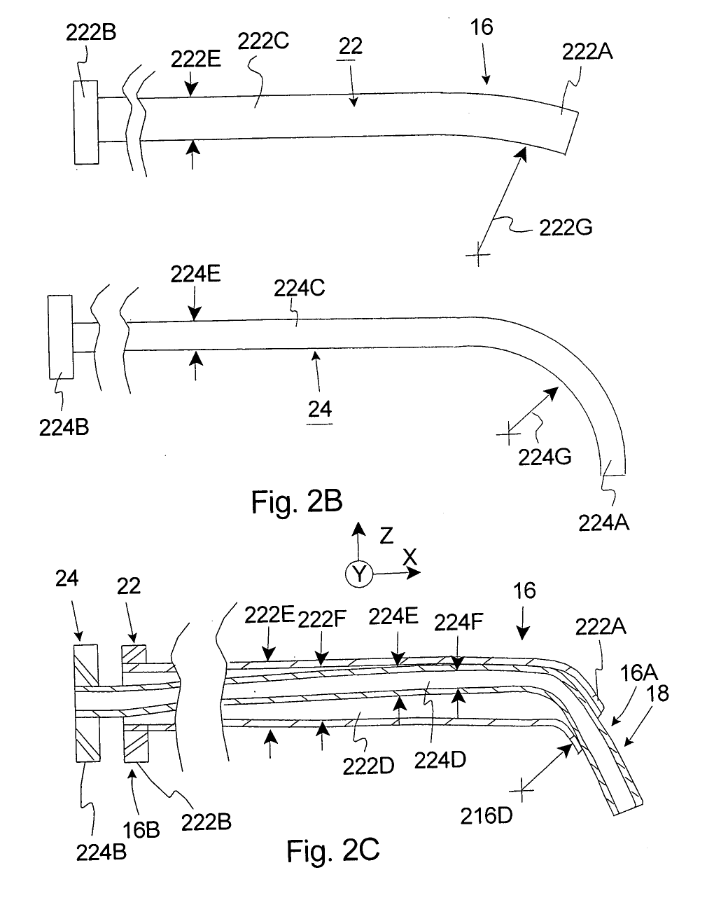

FIG. 2A is a simplified side view, FIG. 2B is a simplified exploded view, and FIG. 2C is a simplified cut-away view of the guide catheter 16, including the first catheter 22 and the second catheter 24. In addition to the catheter tip 16A and the exposed end 16B, the guide catheter 16 can include a generally straight region 216C.

The design of the catheters 22, 24 can be varied pursuant to the teachings provided herein. In one embodiment, the first catheter 22 includes a tubular first distal end 222A that is sized and shaped to be positioned within the vessel 12 (illustrated in FIG. 1), a tubular first exposed end 222B that is designed to be positioned outside the patient 14 (illustrated in FIG. 1), and a tubular first straight region 222C positioned there between. Further, the first catheter 22 can define a first lumen 222D that is sized and shaped to receive the second catheter 24.

Somewhat similarly, the second catheter 24 includes a tubular second distal end 224A that is sized and shaped to be positioned within in the first catheter 22, a tubular second exposed end 224B that is designed to be positioned outside the patient 14, and a tubular second straight region 224C positioned there between. Further, the second catheter 22 can define a second lumen 224D that is sized and shaped to receive a guide wire and other treatment devices.

The size, shape and materials used in the catheters 22, 24 can be varied to provide the desired range of adjustment. For example, the catheters 22, 24 are sized and shaped (i) so that the catheters 22, 24 can be moved concurrently relatively easily together within the vessel 12 (illustrated in FIG. 1) and (ii) so that the catheters 22, 24 can be moved relatively easily relative to each other to adjust and alter the stiffness and/or the shape of the catheter tip 16A. In one non-exclusive embodiment, the first catheter 22 has a first outer diameter 222E of between approximately 0.077 inches and 0.104 inches, and a first inner diameter 222F of between approximately 0.070 inches and 0.090 inches; the second catheter 24 has a second outer diameter 224E of between approximately 0.068 inches and 0.070 inches, and a second inner diameter 224F of between approximately 0.066 inches and 0.070 inches; and the first inner diameter 222F is between approximately 0.002 inches and 0.01 inches greater than the second outer diameter 224E. However, other diameters can be utilized.

The lengths of the catheters 22, 24 can be designed to suit the distance of travel in the vessel 12 to reach the treatment site 12D (illustrated in FIG. 1). For example, each of the catheters 22, 24 can have a length of between approximately 100 centimeters and 130 centimeters. However, other lengths can be utilized.

Each of the catheters 22, 24 can be made of a flexible material. Non-exclusive examples of suitable materials for the catheters 22, 24 include polyurethane or other plastic with a metal braid embedded.

In one embodiment, referring to FIG. 2B, prior to inserting the second catheter 24 into the first catheter 22, the first distal end 222A is curved at a first distal curve and the second distal end 224A is curved at a second distal curve that is different than the first distal curve. More specifically, the first distal curve has a first radius 222G and the second distal curve has a second radius 224G that is different than the first radius 222G. For example, the first distal curve can be less than the second distal curve. In this embodiment, the first radius 222G is greater than the second radius 224G. In one non-exclusive example, the first radius 222G is between approximately 6 centimeters and 12 centimeters, and the second radius 224G is between approximately 3 centimeters and 6 centimeters. Alternatively, the distal curves can have other radiuses.

Further, in one embodiment, the first distal end 222A has a first distal stiffness that is different than a second distal stiffness of the second distal end 224A. For example, the first distal stiffness can be greater than the second distal stiffness. In alternative, non-exclusive examples, the first distal stiffness can be 10, 20, 30, 40, 50, 60, 70, 80, or 100 percent greater than the second distal stiffness. Alternatively, the catheters 22, 24 can have other distal stiffnesses or the stiffnesses can be reversed.

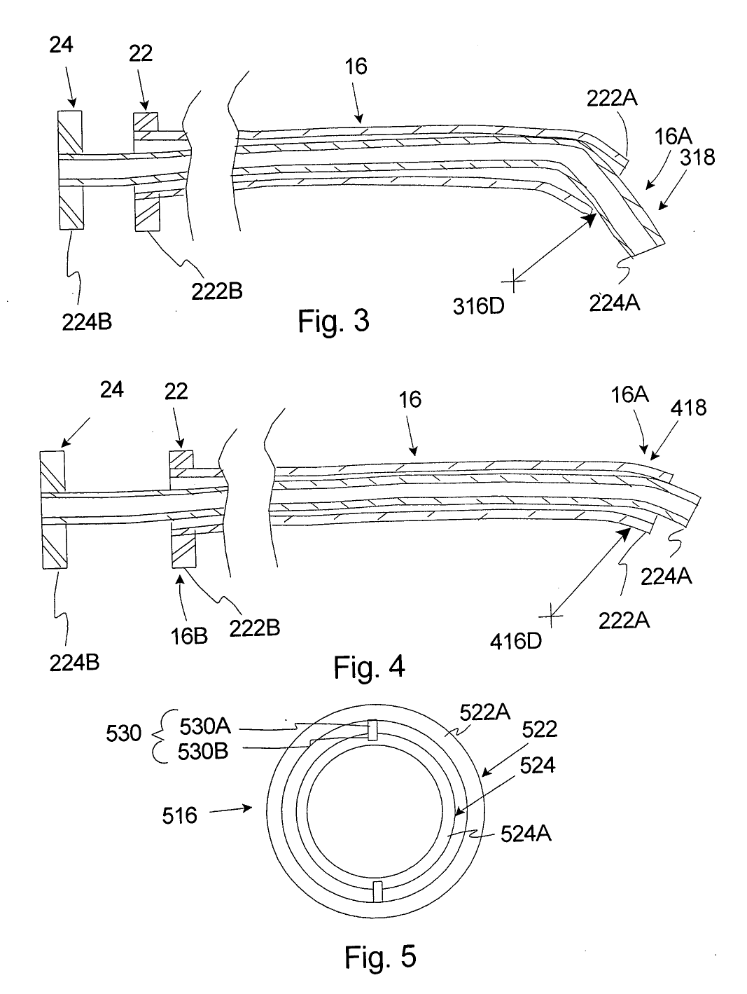

FIG. 2C illustrates the guide catheter 16 in the first configuration 18, FIG. 3 illustrates the guide catheter 16 in a second configuration 318, and FIG. 4 illustrates the guide catheter 16 in a third configuration 418. These configurations 18, 318, 418 are merely examples of possible configurations. It should be noted that other configurations can be achieved.

In certain embodiments of the present invention, relative movement of the catheters 22, 24 changes the configuration of the guide catheter 16, and at least one of the stiffness and the shape of the catheter tip 16A. For example, relative movement of the catheters 22, 24 along an axis (e.g. an X axis illustrated in FIG. 2C) can change at least one of the stiffness and the shape of the catheter tip 16A.

In this embodiment, movement of the second exposed end 224B relative to the first exposed end 222B by the physician causes the second distal end 224A to move relative to the first distal end 222A. Because of the different curves of the distal ends 222A, 224A, relative movement causes the shape of the catheter tip 16A to change. Further, because of the difference in stiffness of the distal ends 222A, 224A, relative movement causes the stiffness of the catheter tip 16A to change. With this design, the physician can selectively adjust the exposed ends 222B, 224B to selectively adjust the shape and stiffness of the catheter tip 16A.

In the first configuration 18, the catheter tip 16A has a first shape with a first tip radius 216D, and a first stiffness. In the second configuration 318, the catheter tip 16A has a second shape with a second tip radius 316D that is different than the first tip radius 216D, and a second stiffness that is different than the first stiffness. In the third configuration 418, the catheter tip 16A has a third shape with a third tip radius 416D that is different than the first tip radius 216D and the second tip radius 316D, and a third stiffness that is different than the first stiffness and the second stiffness.

In one non-exclusive example, the tip radius of the catheter tip 16A is adjustable between approximately 0.5 centimeters and 7 centimeters; and/or the stiffness of the catheter tip 16A is adjustable at least approximately 2, 5, 10, 20, 30, 40, 60, 80, or 100 percent. However, other shapes and stiffnesses can be achieved

FIG. 5 is a simplified end view of another embodiment of a guide catheter 516 having features of the present invention. In this embodiment, the guide catheter 516 again includes a first catheter 522 and a second catheter 524 that are somewhat similar to the corresponding components described above. However, in this embodiment, the guide catheter 516 includes a rotation inhibitor 530 that inhibits relative rotation between the first distal end 522A and the second distal end 524A. With this design, relative movement between the first catheter 522 and the second catheter 524 is limited to sliding along a first axis. This simplifies the control of the stiffness and the shape of the catheter tip 516A.

The design of the rotation inhibitor 530 can vary. For example, the rotation inhibitor 530 includes a generally rectangular shape guide 530A and a corresponding guide channel 530B. In FIG. 5, the guide 530A is fixed to the first catheter 522 and the guide channel 530B is a rectangular shaped groove in the second catheter 524 that receives the guide 530A. Alternatively, these components can be switched or the rotation inhibitor 530 can have a different design.

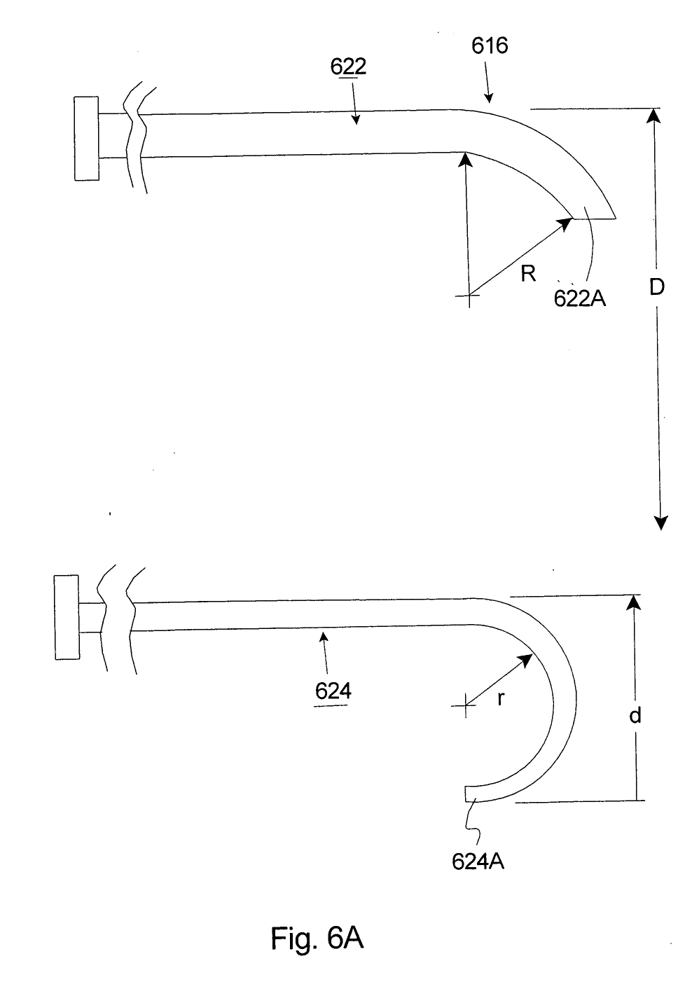

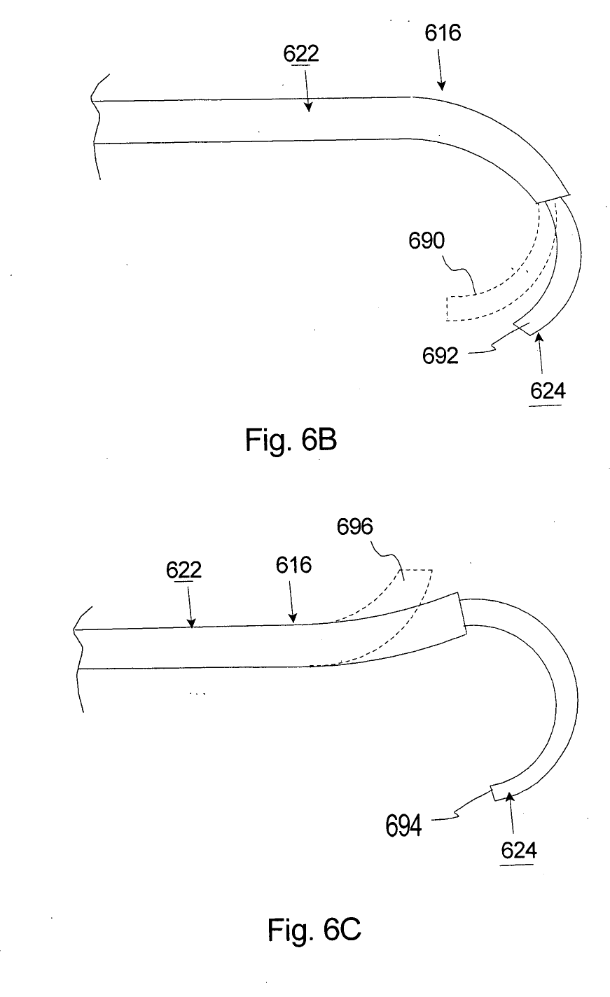

FIG. 6A is a simplified exploded view of another embodiment of a guide catheter 616 that includes a first catheter 622 and a second catheter 624 that fits and moves within the first catheter 622. In this embodiment, the first catheter 622 and the second catheter 624 are somewhat similar to the corresponding components described above. However, in this embodiment, the first distal end 622A and the second distal end 624A are somewhat different than those illustrated in the previous Figures.

More specifically, in FIG. 6A, the first distal end 622A of the outer first catheter 622 has the shape of a tube that is bent to approximately form one eighth of a tubular ring that has a radius of “R” and an effective diameter of “D”. Further, the second distal end 624A of the inner second catheter 624 has the shape of a tube that is bent to form one half of a tubular ring having a radius “r” and a diameter “d”. In this embodiment, r is less than R and d is less than D. In one non-exclusive embodiment, D=2 d and R=2 r. Thus, the radius R of the first distal end 622A is two times greater than the radius r of the second distal end 624A. In this embodiment, d can be equal to between approximately 1-8 centimeters while D can be equal to between 2-16 centimeters. More specifically, in one embodiment d can be equal to approximately 3.5 centimeters while D can be equal to approximately 7 centimeters. Is should be noted that other ratios of R to r can be utilized. For example, R can be approximately 3, 2.5, 2.2, 1.8, or 1.5 r.

Additionally, in this embodiment, the second catheter 624 can be longer than the first catheter 622. For example, the second catheter 624 can be at least approximately five centimeters longer than the first catheter 622

FIG. 6B is a simplified side view of the guide catheter 616 of FIG. 6A in a first configuration and FIG. 6C is a simplified side view of the guide catheter 616 of FIG. 6A is a second configuration. These Figures illustrate two possible ways that the catheters 622, 624 can be manipulated to change the shape and/or stiffness of the distal tip of the guide catheter 616. For example, the inner second catheter 624 changes shape as the outer first catheter 622 is slid over it. FIG. 6B illustrates the original configuration 690 (before the first catheter 622 is slid over it) of the second catheter 624 in phantom, and the first configuration 692 of the distal tip of the guide catheter 616 that results from the first catheter 622 being advanced over the second catheter 624.

FIG. 6C illustrates the resulting second configuration 694 that results from the first outer catheter 622 being rotated one hundred and eighty degrees relative to the second inner catheter 624. This configuration 694 can be used for opposition of the contralateral wall of the aorta. For example, as the outer catheter 622 is rotated relative to the inner catheter 624, the inner catheter 624 will bend away towards the contralateral wall of the aorta. FIG. 6C also illustrates the original configuration 696 in phantom of the outer first catheter 622.

With reference to all of the Figures, one, simplified, non-exclusive method for using the guide catheter 16 includes the steps of: (i) taking an x-ray on the patient to locate and evaluate the treatment site 12D, (ii) inserting a sheath 25 into the vessel 12 in the groin or arm, (iii) inserting the catheter tip 16A into a lumen of the sheath 25, (iv) moving the catheter tip 16A through the blood vessel into the aorta 12A of the heart until the catheter tip 16A is positioned in the ostium 12C of one of the coronary arteries 12B, (v) moving a guide wire through the guide catheter 16 until the guide wire is positioned in the coronary artery past the blockage 12D, (vi) moving a balloon catheter through the guide catheter 16 and over the guide wire until the balloon is positioned at the blockage 12D, (vii) expanding the balloon to open the blockage 12D, (viii) deflating the balloon, and (ix) sequentially removing the balloon catheter, the guide wire, the guide catheter, and the sheath from the patient.

It should be noted that while moving the catheter tip 16A through the blood vessel, the physician can selectively adjust the shape and/or the stiffness of the catheter tip 16A as needed to facilitate movement of the catheter tip 16A.

In one embodiment, because the inner catheter is longer than the outer catheter, one can slide the outer catheter forward, on already placed in the ascending aorta inner catheter. Depending on the anatomy of aorta and coronary ostia, the final shape of the guide catheter can be produced by sliding forward and backwards the outer catheter over the inner catheter till an appropriate shape for coronary ostium engagement is produced. Once the ostium is intubated, the procedure can proceed in usual fashion.

If more support is needed from the guide catheter, then the outer catheter can be used to stiffen the system by pushing it forward, over the fixed inner catheter and the equipment that is introduced into the coronary through it. If the shape shift of the distal part of the system produced by this move causes unfavorable change in the angle between its tip and the intubated coronary artery, then in order to stiffen the system one might pull back the outer catheter so that its curved part approaches the straight distal part of the inner catheter, and then rotate the outer catheter 180 degrees on the fixed inner catheter. This move will cause the system to better oppose the contralateral wall of the ascending aorta, increasing the guide catheter support. This is illustrated in FIG. 6C.

Further, while the particular guide catheter 16 as shown and disclosed herein is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A guide catheter for movement within a vessel of a mammal, the guide catheter comprising:
a tubular catheter assembly including an exposed end that is positioned outside the vessel, and a catheter tip that is movable within the vessel with manipulation of the exposed end; wherein at least one of a stiffness and a shape of the catheter tip can be selectively adjusted between a first configuration and a second configuration with the exposed end.

2. The guide catheter of claim 1 wherein both the stiffness and the shape of the catheter tip can be adjusted with the exposed end.

3. The guide catheter of claim 1 wherein the catheter assembly includes a first catheter and a second catheter designed to be moved together within the vessel, the first catheter including a tubular first distal end that is sized and shaped to be positioned within the vessel, the second catheter including a tubular second distal end that is sized and shaped to be positioned within in the first catheter; wherein relative movement of the catheters changes at least one of the stiffness and the shape of the catheter tip.

4. The guide catheter of claim 3 wherein movement of the catheters changes both the stiffness and the shape of the catheter tip.

5. The guide catheter of claim 3 wherein the first distal end is curved at a first distal curve and the second distal end is curved at a second distal curve that is different than the first distal curve.

6. The guide catheter of claim 5 wherein the first distal curve is less than the second distal curve.

7. The guide catheter of claim 5 wherein the first distal curve has a first radius that is greater than a second radius of the second distal curve.

8. The guide catheter of claim 3 further comprising a rotation inhibitor that inhibits relative rotation between the first distal end and the second distal end.

9. The guide catheter of claim 1 wherein a shape of the catheter tip is adjustable between a tip radius of between approximately 0.5 centimeters and 7 centimeters.

10. The guide catheter of claim 1 wherein a stiffness of the catheter tip is adjustable at least approximately 2 percent.

11. A guide catheter for movement within a vessel of a mammal, the guide catheter comprising:
a tubular catheter assembly including an exposed end that is positioned outside the vessel, and a catheter tip that is movable within the vessel with manipulation of the exposed end; wherein the catheter assembly includes a first catheter and a second catheter designed to be moved together within the vessel, the first catheter including a tubular first distal end that is sized and shaped to be positioned within the vessel, the second catheter including a tubular second distal end that is sized and shaped to be positioned within in the first catheter; wherein prior to assembly of the catheters, the first distal end is curved at a first distal curve and the second distal end is curved at a second distal curve that is different than the first distal curve.

12. The guide catheter of claim 11 wherein the first distal curve is less than the second distal curve.

13. The guide catheter of claim 11 wherein the first distal curve has a first radius that is greater than a second radius of the second distal curve.

14. The guide catheter of claim 11 further comprising a rotation inhibitor that inhibits relative rotation between the first distal end and the second distal end.

15. The guide catheter of claim 11 wherein a shape of the catheter tip is adjustable between a tip radius of between 0.5 centimeters and 7 centimeters.

16. The guide catheter of claim 11 wherein a stiffness of the catheter tip is adjustable at least approximately 5 percent.

17. A method for moving a guide catheter through a vessel of a mammal, the method comprising the steps of:
providing a tubular catheter assembly including an exposed end that is positioned outside the vessel, and a catheter tip that is movable within the vessel with manipulation of the exposed end; and
selectively adjusting at least one of a stiffness and a shape of the catheter tip while the catheter tip is positioned in the vessel.

18. The method of claim 17 wherein the step of selectively adjusting includes selectively adjusting both the stiffness and the shape of the catheter tip.

19. The method of claim 17 wherein the step of providing a tubular catheter assembly includes providing a first catheter and a second catheter designed to be moved together within the vessel, the first catheter including a tubular first distal end that is sized and shaped to be positioned within the vessel, the second catheter including a tubular second distal end that is sized and shaped to be positioned within in the first catheter; and wherein the step of selectively adjusting includes the step of moving the catheters relative to each other to change at least one of the stiffness and the shape of the catheter tip.

20. The method of claim 19 wherein the first distal end is curved at a first distal curve and the second distal end is curved at a second distal curve that is different than the first distal curve.
